(19) United States
(12) Patent Application Publication
(10) Pub. No.: US 2009/0203646 A1
(43) Pub. Date: Aug. 13, 2009

(54) USE OF SODIUM NARCISTATIN FOR REDUCING INTERNAL ADHESIONS AND FIBROSIS

(75) Inventors: Cheri Lubahn, Surprise, AZ (US); Dianne Lorton, North Royalton, OH (US)

Correspondence Address: JENNINGS, STROUSS & SALMON, P.L.C., 201 E. WASHINGTON ST., 11TH FLOOR, PHOENIX, AZ, 85004, US

(73) Assignee: Sun Health Research Institute

(21) Appl. No.: 12/370,535

(22) Filed: Feb. 12, 2009

Related U.S. Application Data
(60) Provisional application No. 61/028,173, filed on Feb. 12, 2008.

(51) Int. Cl. A61K 31/675; A61K 31/4355; A61P 1/04

(52) U.S. Cl. 514/81; 514/287

(57) ABSTRACT

The present invention concerns a method for reducing or preventing development of adhesions, fibrosis or scar tissue within serous body cavities by using sodium narcistatin.

### CROSS REFERENCE TO RELATED APPLICATIONS

This application claims the benefit of priority of U.S. Provisional Application Ser. No. 61/028,173, filed Feb. 12, 2008.

### FIELD OF THE INVENTION

The present invention relates to methods for the reduction or prevention of adhesions, fibrosis and scar tissue that occurs in, but is not limited to, rheumatoid arthritis, other inflammatory autoimmune diseases, chronic inflammation, post-surgical procedures, infection and exposure to toxins, and more specifically, to the use of sodium narcistatin in reducing and preventing development of internal adhesions, fibrosis and scar tissue.

### BACKGROUND OF THE INVENTION

Adhesions are a significant complication of surgery, infection, and chronic inflammatory disorders, such as rheumatoid arthritis. Other recognized causes of adhesions include bacterial peritonitis, radiotherapy, chemical peritonitis, foreign body reaction, long-term continuous ambulatory peritoneal dialysis, endometriosis and pelvic inflammatory disease.

Adhesions develop in 93% of patients that undergo abdominal and pelvic surgery. Examples of abdominal and pelvic surgery include hernia repair, gynecological surgeries, colorectal surgeries, and many others. Five to 60% of poor surgical outcome after or compressive spine surgery results from excessive post-operative fibrosis. Between 50 to 95% of women undergoing gynecological surgery develop adhesions.

Adhesions of the peritoneal cavity can lead life-threatening complications such as pancreatitis, diabetes, hepatitis, cirrhosis, liver failure, and bowel obstruction. Formation and reformation of intra-abdominal adhesions after surgery is a significant cause of morbidity. Intra-abdominal adhesions after surgery results in 33% of patients being readmitted an average of twice to hospitals for related complications. While congenital or inflammatory adhesion rarely give rise to intestinal obstruction, post-operative adhesion are responsible for 40% of all intestinal obstruction cases and 60 to 70% of ones that involve the small bowel.

The presence of adhesions during second surgeries to ameliorate complications that occur after the first surgery often result in longer operating times and are at increased risk for intra-operative complications, including bowel, bladder, ureters, and bleeding. Intra-pelvic adhesions can result in infertility by forming a mechanical blockage to the fallopian tubes, thus preventing oocyte retrieval.

Adhesions of the internal organs also can cause pain and discomfort. Division of adhesions at surgery has been shown useful in treatment of chronic pelvic pain. In RA fibrosis of the heart and lungs and/or development of abdominal adhesions involving the kidney, intestinal tract and spleen have all been associated with the disease development and increased risk of morbidity. RA is a systemic inflammatory disease that can involve other tissues and organs as well as synovial joints. Involvement of these extra-articular features and non-articular complications of RA are common and are generally related to worse disease and elevated risk for mortality.

In RA, adhesions observed in peritoneal and thoracic viscera formed during disease development are very painful, inhibit adequate blood flow, constrict organ motility, cause metabolic disregulation and contribute to morbidity. Thus, there is a great need to recognize these complications early to promptly provide appropriate care. Chronic infection and toxicity also have been associated with fibrosis and/or adhesions of the inter-peritoneal organs.

### BRIEF DESCRIPTION OF THE DRAWINGS

FIG. 1 illustrates the chemical structure of compounds of the present disclosure.

FIG. 2 is a micrograph demonstrating the extensive adhesions present surrounding the spleen in the peritoneal cavity in rats with inflammatory arthritis that were treated with the vehicle for sodium narcistatin and the lack of splenic adhesions in rats with inflammatory arthritis that were treated with sodium narcistatin.

FIG. 3 is a graph demonstrating that reduced splenic adhesion formation the peritoneal cavity in rats with inflammatory arthritis that were treated with the vehicle for sodium narcistatin and the lack of splenic adhesions in rats with inflammatory arthritis that were treated with sodium narcistatin.

FIG. 4 is a graph demonstrating the effect of sodium narcistatin on inflammatory cytokine production by immune cells obtained from the spleen and treated with the T-cell dependent mitogen, Con A.

FIG. 5 is a graph demonstrating the effect of sodium narcistatin on inflammatory cytokine production by immune cells obtained from the spleen and treated with the B-cell dependent mitogen, LPS.

FIG. 6 is a graph demonstrating that sodium narcistatin inhibits fibroblast proliferation in vitro.

### DETAILED DESCRIPTION OF VARIOUS EMBODIMENTS

This invention relates to the prevention of internal adhesions that result from but not limited to chronic inflammatory conditions, surgical interventions, infections and toxin exposure. The invention relates to prevention of adhesion development in serous cavities including the peritoneum, the pericardium, the pleura and synovial cavities such as joints and tendons and to adhesion following spinal and/or cranial operations.

At present, there are no ideal methods of preventing adhesion formation/reformation. Multiple agents have been evaluated for their ability to prevent and reduce adhesion development. This includes agents that inhibit inflammation (examples include both steroidal and non-steroidal anti-inflammatory medications), agents that degrade fibrin (such as recombinant tissue plasminogen activator), and barrier methods involving the application of an absorbable material/solution/gel intraperitoneally to prevent adherence of peritoneal organ surfaces to one another. However, none of these eliminates adhesion formation/reformation.

Oxidized regenerated cellulose (Interceed) and polytetrafluoroethylene (PTFC, Gore-Tex) are commercially available synthetic barriers for adhesion prevention. However, the Interceed Absorbable Adhesion Barrier (Ethicon, Inc., Somerville, N.J., USA) must be placed onto the raw tissue area after adequate hemostasis is achieved. In contrast, Gore-Tex (WL Gore & Associates, Inc., Flagstaff, Ariz., USA) is non-absorbable and needs to be sutured at the surgical site. It also has the disadvantage that it must be removed during a subsequent surgical procedure. Both of these barriers do not eliminate the formation and reformation of adhesion in all patients.

Cellular Events Involved in Peritoneal Healing and its Relevance to the Pathogenesis of Adhesion Formation/Reformation

Serous membranes that line the peritoneal, pleural, and pericardial cavities have similar embryological precursors. Serous membranes are lined by a layer of mesothelial cells which are anchored to the basement membrane. The submesothelial layer is made up of extracellular matrix (ECM) composed of different types of collagen, glycoproteins, (i.e. laminin and fibronectin), glycosaminoglycans and proteoglycans. Blood and lymphatic vessels travel in the subserous space. Fluid diffusion and resorption freely occurs through the mesothelium and submesothelial stroma.

Slight trauma to the mesothelial cells can cause these cells to be easily detached from the basement membrane. After such trauma, these cells play a key role in the physiology of adhesion development by secreting proinflammatory cytokines (interleukin (IL)-1, IL-6, IL-8, tumor necrosis factor (TNF)-alpha) and transforming growth factor (TGF)-beta, hyaluronic acid and prostaglandins when stimulated in vitro. These cells contribute to the fibrinolytic process by secreting tissue plasminogen activator and expressing intracellular adhesion molecule-1 (ICAM-1).

After trauma/injury to the peritoneum, vascular permeability increases in vessels that supply the damaged area, followed by an exudation of inflammatory cells. The peritoneal leukocytes, mesothelial cells, and macrophages produce cellular mediators, which severe to modulate and orchestrate the subsequent response of other cells involved in the inflammatory response. This ultimately leads to the formation of a fibrin matrix. The fibrin matrix is gradually organized and replaced by tissue containing fibroblasts, macrophages and giant cells. This matrix forms fibrin bands, which connect two injured peritoneal surfaces together. Fibrin bands can be broken down into smaller molecules (fibrin degradation products) by fibrinolysis.

With fibrinolysis, fibrin clots that are formed during the healing process are broken down. In this process, plasminogen is converted to plasmin by the serine proteases, tissue plasminogen activator (tPA) and urokinase-like plasminogen activator uPA). Plasmin, whose main role is to degrade fibrin, can be produced by macrophages or mesothelial cells lining the peritoneal cavity. The activity of tPA and uPA to break down fibrin clots can be inhibited by plasminogen activator inhibitor-1 (PAI-1) and PAI-2. In the process of adhesion formation, the fibrinolytic process is altered, allowing the formation and persistence of adhesions.

In conditions of aberrant peritoneal healing, fibrinolysis is reduced as a result of ischemia and the fibrin bands persist. Over-time the fibrin bands become organized leading to persisting adhesions. The initial adhesion tissue is a mixture of macrophages, eosinophils, red blood cells, tissue debris, mast cells and fibroblasts, but within 5-7 days after injury the majority of cells are fibroblasts.

The activity of peritoneal plasminogen activators is significantly reduced. This is in part due to increased peritoneal concentration of PAI. Further, individuals undergoing surgery that developed severe adhesions were found to overexpress PAI-1 and had reduced tPA activity, not only in adhesion tissue, but also in the peritoneal tissue adjacent to the sites of the adhesions. These findings suggesting that post-operative adhesion tissue has a reduced ability to degrade fibrin.

Proteases and Protease Inhibitors

Other proteinases, particularly the metalloproteinases (MMP) and their inhibitors: tissue inhibitors of metalloproteinases (TIMP), also interact with the fibrinolytic system and are important players in the remodeling of the ECM after injury of internal tissues. MMPs are a family of enzymes that can degrade ECM components. The actions of MMP to degrade ECM are inhibited by TIMP. Collectively, MMPs are capable of degrading all of the components of the ECM. Aberant MMP and TIMP expression has been associated with adhesion formation/reformation.

After trauma/injury to the peritoneum, plasmin can activate latent MMP. Surgical manipulation that results in adhesion formation has been shown to alter both the PA/PAI and MMP/TIMP equilibrium in the peritoneal fluid. Treatment with a gonadotrophin-releasing hormone (GnRH) agonist reduced adhesion formation after surgery, an effect that was associated with decreased PA and MMP activity and increased PAI and TIMP activity in the peritoneal fluid. The GnRH-induced altered balance between the PA/PAI and the MMP/TIMP system was postulated to cause a shift to a less invasive phenotype that alters fibrinolysis and ECM remodeling.

Role of Transforming Growth Factor (TGF-β)

Plasmin produced during peritoneal healing and adhesion formation also activates latent TGF-β. Activated TGF-β interacts not only with the fibrinolytic system and ECM but also with many other cellular mediators involved in the process of adhesion formation. TGF-β is a key player in normal wound healing and a potent inducer of tissue fibrosis in peritoneal wound healing. During the acute phase of the inflammatory response, peritoneal macrophages and/or mesothelial cells produce TGF-β that contribute to the synthesis of the ECM by stimulating fibroblastic cell production of collagen and fibronectin.

Overexpression of TGF-β by cells in the parietal peritoneum and serosal surfaces of the pelvic organs and increased TGF-β levels in the peritoneal fluid are associated with increased incidence of adhesion formation in humans and animals. TGF-β has been shown to increase PAI-1 mRNA and decreased tPA mRNA expression in vitro. Addition of TGF-β to human mesothelial cells decreased MMP-1 but increased TIMP-1 mRNA expression in vitro. Thus, TGF-β can differentially regulate MMP and TIMP, PAI and PA at the transcription level to alter their production and ultimately the production of ECM and therefore, exerts substantial influence over the outcome of peritoneal healing and adhesion formation.

Role of Pro- and Anti-Inflammatory Cytokines (TNF-α, IL-1, IL-6, IL-8, IL-10 & IFN-γ)

Cytokine profiles are altered in the peritoneal fluid in the presence of adhesions. With inflammation of the peritoneum after peritoneal injury, the peritoneal fluid has increased concentrations of the pro-inflammatory cytokines, TNF-α, IL-1, IL-16, and IL-8. TNF-α, IL-1 and IL-6 are known to interact with the fibrinolytic system to affect adhesion formation. Plasmin causes mobilization and release of TNF-α, IL-1, and IL-6. Both TNF-α and IL-1β can subsequently reduce the expression of tPA at both the protein and mRNA level.

In this manner, these cytokines play a role in regulating not only the inflammatory response after injury, but also the extent and severity of adhesion formation and reformation. While there are significant controversies regarding the concentrations of a number of cytokines in the peritoneal fluid of subjects with well-established adhesion, some studies have found elevated TNF-α levels in the serum and peritoneal fluid of patients with adhesions during surgery.

Administration of IL-1 into the peritoneal cavity contributes to adhesion formation in experimental rat models. Similarly, IL-6 has peritoneal adhesion promoting effects. IL-6 administration into the peritoneal cavity of rats increased adhesion formation significantly. Further, elevated levels of IL-6 correlate with non-endometriotic pelvic adhesions.

Anti-inflammatory cytokines also are involved in peritoneal adhesion formation. Low levels of IL-10 and IFN-γ are reported in the peritoneal fluid of subjects with adhesion or endometriosis. Low levels of IL-10 also were found in the peritoneal fluid after surgery in rats. Administration of IL-10 into the peritoneal cavity reduces adhesions in rats after surgery.

Selective immunosuppression has been used to reduce adhesion formation/reformation. Rats treated intraperitoneally with antibodies against TGF-β1, IL-1 or TNF-α developed comparatively fewer adhesions than the control rats. The fewest adhesions were observed when a combination of anti-IL-1 and anti-TNF-α antibodies were administered together. Pre-operative treatment with anti-IL-6 antibodies given i.p. also reduces adhesion formation.

Treatment with the anti-inflammatory cytokine IL-10 limited post-operative intraperitoneal adhesion formation without significant systemic effects. Similarly, IL-10 and/or Ketorolac (a non-steroidal anti-inflammatory drug) reduces adhesion formation and also resulted in thinner and filmier adhesions. In contrast, systemic immunosuppression by treatment of rats with cyclosporine did not reduce adhesion formation/reformation.

Collagen Deposition, Adhesions and Fibrosis

Adhesions are fibrous bands that connect tissue surfaces that are normally separated. Adhesion/scar formation is a normal consequence of surgery or other tissue injury and is required for proper wound healing. Despite the role of scar tissue in the normal healing process, in many cases, the adhesions/scar tissue overgrows the intended region and creates pathological adhesions that are detrimental to normal body functions and that can cause significant complications for the patient. For example, following an abdominal surgery, adhesions may form between an incision in the abdominal wall and the small bowel, causing an obstruction in the small bowel. This type of adhesion often leads to additional surgical procedures to remove the obstruction.

Fibrosis results from the infiltration and proliferation of fibroblasts into an area of tissue injury. These fibroblasts then produce a collagen rich extracellular matrix that interferes with the proper function and communication of the tissue's cells. In this specific aim we will use a traditional in vitro model of fibrosis to demonstrate SNS's anti-proliferative and anti-fibrosis effects on fibroblasts.

The present disclosure provides for a novel drug, sodium narcistatin (SNS) and method for using the drug in a manner which exhibits anti-fibrosis and anti-adhesive properties when used as an anti-inflammatory and joint sparing treatment in an animal model, and by extension to human animals, for rheumatoid arthritis (RA). Treatment with SNS effectively inhibited proliferation of cells associated with development of fibrosis and adhesions in an animal model of RA. The present disclosure is further concerned with the prevention of the occurrence of adhesions.

In each of the various aspects and embodiments of the disclosure described below, the term “subject” refers to a mammal, preferably a human subject. In each of the various aspects and embodiments of the disclosure described below, the phrase “an effective amount” is an amount that is sufficient to provide the intended benefit of treatment.

In each of the various aspects and embodiments described below, the term “treat” or “treating” means accomplishing one or more of the following: (a) reducing the severity or the disorder; (b) limiting or preventing development of symptoms characteristic of the disorder(s) being treated; (c) inhibiting worsening of symptoms characteristic of the disorder(s) being treated; (d) limiting or preventing recurrence of the disorders(s) in patients that have previously had the disorder(s); and (e) limiting or preventing recurrence of symptoms in patients that were previously symptomatic for the disorder(s).

The present disclosure provides novel methods for reducing or preventing internal adhesions by treating with sodium narcistatin. The present disclosure addresses one or more short-comings or disadvantages in the available treatment regimens for preventing development of unwanted adhesion, fibrosis and scar tissue that result from a number of conditions, included but not limited to chronic inflammation, surgical procedures, infection and exposure to toxins through the use of sodium narcistatin.

Amaryllidaceae alkaloids: SNS is a derivative of an Amaryllidaceae alkaloid, narciclasine. Amaryllidaceae alkaloids are derived from constituents from the bulb of the tropical spider lily plant (Hymenocallis littoralis; Amaryllidaceae). Narciclasine proved to be relatively insoluble in aqueous solutions. An efficient procedure was found for the synthetic synthesis of biologically active narciclasine and a water-soluble derivative that retains its biological activity called sodium narcistatin. This agent has the advantage that it does not need to be directly applied to raw tissue surface to prevent adhesions and could be used even after surgery to prevent adhesion development.

In preferred embodiments, the disclosure contemplates the use of Amaryllidaceae alkaloids and derivative of Amaryllidaceae alkaloids, particularly narciclasine, pancratistatin and narcistatin, as agents to reduce or prevent adhesions, fibrosis and scar tissue formation in conditions that development of adhesions, fibrosis and scar tissue cause medical complications. Amaryllidaceae alkaloids are particularly useful in reducing and preventing development of adhesions, fibrosis and scar tissue and thus prevent medical complications arising from adhesions, fibrosis and scar tissue.

In certain embodiments, the present disclosure concerns a method for preventing/reducing adhesions, fibrosis and scar tissue development by the application of a therapeutically effective dose of a Amaryllidaceae alkaloids, such as sodium narcistatin to human subjects with the diseases or surgical interventions that are known by those skilled in the art to have adhesion, fibrosis and/or scar tissue-induced medical complications. As used herein, the term “reducing or preventing development of adhesions, fibrosis and/or scar tissue by the application of a therapeutically effective dose of a Amaryllidaceae alkaloid” is used to signify that the Amaryllidacea alkaloid is supplied to the patient in amounts, and for a period of time, that are effective to reduce or prevent development of adhesions, fibrosis or scar tissue based on improvement in one or more of the clinically measured parameters of pathological adhesion, fibrosis or scar tissue development.

Amaryllidacea alkaloids may be administered to the patient in any pharmaceutically acceptable vehicle and by any route heretofore acceptable for these agents. The preferred route of administration is orally, although one may, if desired, choose to administer the Amyaryllidacea alkaloids intravenously, sublingually, intramuscularly, subcutaneously, topically or in a sustained release form.

As will be understood by those skilled in the art, the effective doses of the Amaryllidacea alkaloids will depend upon the route of administration and the patient's sensitivity to the particular Amaryllidacea alkaloid. Recommended doses for the Amaryllidacea alkaloids range from about 2.5 to 5 mg/Kg body weight given two times per day depending upon condition for which the drug will be used to reduce or prevent adhesions, fibrosis or scar tissue development and patient responses to the drugs. The dosages may be more effectively adjusted on an individual basis as adhesion, fibrosis, and scar tissue development varies from patient to patient and as the risk of adhesion, fibrosis, and scar tissue development varies between conditions.

In each of the various aspects and embodiments described below, the terms “narcistatin”, “pancratastatin”, pancratastatin-7′ phosphate”, and pancratastatin-3,4′ cyclic phosphate” include cations thereof. Such cations include, but are not limited to H+, Li+, Na+, K+, Cs+, Mg2+, Ca2+, Zn2+, Mn2+, pyridinium, quinidine, quinine, imidazole, morpohiline, and piperazine.

Narciclasine and several related isocarbostyrils isolated from, for example, the bulbs of Narcissus and Hymenocallis species (Amaryllidaceae) have been found to possess anticancer properties. Narciclasine has poor solubility which could be advantageous in treating some conditions where pathological adhesions occur. Sodium narcistatin is a synthetic modification of narciclasine that is highly water soluble. Pancratastatin, another compound derived from Hymenocallis Littoralis has been well-characterized, and appears to be more potent than sodium narcistatin in inhibiting tumor growth. Pancratastatin has been found to increase survival rate up to 100% against a flavivvirus infection, Japanese encephalitis and to have activity against the parasite Encephalitozoan intestinalis, a microsporidian causing intestinal and systemic infections in immunocompromised patients

Like narcicalsine, pancratastatin also has relatively low solubility in biological fluids. As a result, the phsophorylated analog pancratastatin-7″-phosphate was developed. A further derivative of the pancratastatin series is pancratastatin-3′,4′-cyclic phosphate. Each of these compounds share similarity to sodium narcistatin, including its solubility in biological fluids. Given their similar structure and their comparable inhibition of similar biological functions between narcistatin and pancratastin, we predict that the derivative should bear activities similar to those of narcistatin demonstrated herein.

In the present methods for preventing adhesions, fibrosis and/or scar tissue, the type of adhesions, fibrosis and or scar tissue can be any type of adhesions, fibrosis or scar tissue, but resulting preferably from conditions of chronic inflammation, surgical interventions, infection, and exposure to toxins, including but not limited to rheumatoid arthritis, abdominal and pelvic surgeries and alcohol abuse.

An effective amount of the compounds that can be employed ranges generally between about 0.01 μg/Kg body weight and about 20 mg/Kg body weight, preferably ranging between about 0.05 μg/Kg and about 10 mg/Kg body weight. However dosage levels are based on a variety of factors, including the type of injury, age, weight, sex, medical condition of the individual, the severity of the condition, the route of administration, and the particular compound employed. Thus, the dosage regimen may vary, but can be determined routinely using standard methods.

The data disclosed herein provide evidence that narcistatin, and related compounds, are effective anti-inflammatory agents and anti-fibroblast proliferation agents that are effective in reducing and/or preventing adhesions, fibrosis and scar tissue formation in such disorders as pathological internal adhesions. Without being limited by any specific mechanism, the inventors believe that the various therapeutic effects of narcistatin and related compounds disclosed herein may be due, at least in part, to the inhibition of proinflammatory cytokine production and anti-proliferative effect on fibroblasts.

Herein is provided a method of preventing or reducing the incidence of adhesions that result from chronic inflammation that occur in a body cavity of a subject, which comprises administering to a subject with a potential to develop internal adhesions or with internal adhesions an effective amount of a compound selected from the groups consisting of sodium narcistatin, or structurally related compounds including narciclasine, pancratastatin pancratastatin-7′ phosphate and pancratastatin-3′,4′ cyclic phosphate, or pharmaceutically acceptable salts thereof.

The process of developing adhesions, fibrosis, and scar tissue is central to a number of disease states, including chronic inflammatory conditions, and with exposure to toxins and surgical conditions. Development of adhesions, fibrosis and scar tissue involves an orchestrated series of events initiated in response to tissue damage that is briefly described in the background for the invention. A method of preventing or reducing the incidence of internal adhesion and/or fibrosis in or associated with a body cavity, which comprises treatment with sodium narcistatin or structurally related compounds as defined above.

The sodium narcistatin used in one embodiment of the present disclosure is water soluble, but is expected to be effected in other formulations, such as, but not limited to, formulated within a gel, a tablet or capsule. Other less water soluble structurally related compounds are expected to be more suitable to prevent fibrosis in some conditions to prevent adhesions, fibrosis or scar tissue formation. The compounds may be made up in a solid form (including granules, powders or suppositories) or in a liquid form (e.g., solutions, suspensions, gels, emulsions). The compounds of the disclosure may be applied in a variety of solutions and may be subjected to conventional pharmaceutical operations such as sterilization and/or may contain conventional adjuvants, such as preservatives, stabilizers, wetting agents, emulsifiers, buffers, etc.

The present disclosure also provides a composition comprising an aqueous solution, suspension, gel, capsule, tablet formulation of sodium narcistatin or other structurally related compounds defined above in which the amount of sodium narcistatin or structurally related compound is effective to prevent or reduce the incidence of adhesions, fibrosis or scar tissue associated with chronic inflammation, surgical interventions, infection or toxins.

For administration, the compounds are ordinarily combined with one or more formulation components appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the compounds of this disclosure may be dissolved in saline, water, polyethylene glycol, propylene glycol, carboxymethyl cellulose colloidal solutions, ethanol, tragacanth gum, and/or various buffers. Other formulation components and modes of administration are well known in the pharmaceutical art. The carrier or diluent may include time delay material, such as glyceryl monostearate or glyceryl distearate alone or with a wax, or other materials well known in the art.

The present disclosure further provides the use of sodium narcistatin and other structurally related compounds as defined above in the manufacture of a composition comprising an aqueous solution, suspension, gel, tablet or capsule formulation for preventing or reducing post-operative adhesions in humans and animals.

In one embodiment, a composition of the disclosure is taken orally or applied to the appropriated body cavity or area under situations of injury, as defined above, in which adhesions occur. A composition of the disclosure can be administered during the time frame in which adhesions are known to develop with chronic inflammatory diseases, post-surgery, infections and exposure to toxins. In one embodiment, the composition is administered once daily by an appropriate route of administration for the intended fibrosis inducing condition.

The compounds of the disclosure may be administered by any suitable route, including orally, parentally, by inhalation or rectally in dosage unit formulations containing conventional pharmaceutically acceptable carriers, adjuvants, and vehicles, including liposomes. The term parenteral as used herein includes, subcutaneous, intravenous, intraarterial, intramuscular, intrasternal, intratendinous, intraspinal, intracranial, intrathoracic, infusion techniques, intracavity, or intraperitoneally. In a preferred embodiment, the compounds of the disclosure are administered orally or parentally.

The compound may be administered at once, or may be divided into a number of smaller doses to be administered at intervals of time. It is understood that the precise dosage and duration of treatment is a function of the disease, disorder or condition being treated and may be determined empirically using known testing protocols or by extrapolation from in vivo or in vitro test data.

Preferably a composition of the disclosure should be administered in an effective therapeutic dose for the intended adhesion, fibrosis, or scar tissue inducing condition, ideally over a concentration range of 1 to 10 mg/Kg body weight. In an alternate embodiment of each of the above aspects of the disclosure, the pharmaceutical compositions of the disclosure are prepared for oral or intraperitoneal administration. The pharmaceutical compositions can be in the form of, for example, a tablet, a hard or soft capsule, a lozenge, a cachet, a dispensable powder, granules, a suspension, an elixir, a liquid, or any other form reasonably adapted for oral administration.

The pharmaceutical compositions can further comprise, for example, buffering agents. Tablets, pills and the like additionally can be prepared with enteric coatings. Unit dosage tablets or capsules are preferred. Oral compositions will generally include an inert diluent or an edible carrier and may be compressed into tablets or enclosed in gelatin capsules. Pharmaceutically compatible binding agents and other material known in the art can be included as part of the composition.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Lipsomal suspensions including tissue-targeted lipsomes may also be suitable as pharmaceutically acceptable carriers.

The active compounds may be prepared with carriers that protect the compounds may be prepared against rapid elimination from the body, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

The instant compounds can be administered individually or in combination, usually in the form of a pharmaceutical composition. Such compositions are prepared in a manner well known in the pharmaceutical art and comprise at least one active compound. The compounds of the disclosure can be administered as the sole active pharmaceutical agent, or they can be used in combination with one or more other agents to treat the particular condition. When administered as a combination, the therapeutic agents can be formulated as separate compositions that are given at the same time or different times, or the therapeutic agents can be given as a single composition.

The pharmaceutical compositions of this aspect of the disclosure include mixtures of the compounds of the disclosure, or pharmaceutically acceptable salt thereof, and the one or more other compounds, as well as separate unit dosages of each that are manufactured for combinatorial use. Such separate unit dosages may be administered concurrently or sequentially as determined by the clinician.

The term “pharmaceutically acceptable salts” as used herein in each of the aspects and embodiments of the disclosure refers to those salts that are within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the disclosure. The term “salts” refers to the relatively non-toxic, inorganic and organic acid addition salts of compounds of the present disclosure.

These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumerate, succinate, tartrate, naphthylate mesylate, glucoheptonate, lactobionate, and laurylsulphonate salts, and the like.

These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like.

### EXAMPLE 1

Treatment with SNS dramatically reduced the number of adhesions in the thoracic and abdominal cavities in the arthritic animals compared to arthritic animals that were untreated or treated with the vehicle for SNS.

Animals: Adult, male Lewis rats (weight) were purchased from Charles River Laboratories (Raleigh, N.C.) and housed two per cage. The animals were allowed to adjust to the vivarium at SHRI for 5-7 days prior to experimental use. Rabbits were housed on a 12:12 light:dark cycle with food and water available ad libitum. For AA rats rodent diet (Purina Lab diet 50010 was placed in the bottom of the cage and water was supplied using long-stemmed sipper tubes for easy access to food and water. Other than the development of arthritis, the good health of the animals was maintained throughout the course of the experiments. Dorsoplantar footpad measures mere completed every other day. Prior to sacrifice the animals were given a 1.0 ml intraperitoneal (i.p,) injection of 8% chloral hydrate in sterile saline and radiographs were taken of their hind limbs to assess disease severity. The animals were then sacrificed with an overdose of chloral hydrate.

Arthritis was induced in young adult male Lewis rats by base of the tail intradermal (i.d.) injection of complete Freund's adjuvant (CFA; 0.03 g M. butyricum/emulsified in 10 ml sterile mineral oil). CFA (0.03 g M. butyricum/emulsified in 10 ml mineral oil) was prepared by grinding the M. butyricum with a mortar and pestle until the lyophilized bacteria had turned from a light beige to an eggshell white powder. The mineral oil was slowly worked into the heat killed bacterial cell wall using the mortar and pestle. The suspensions were treated with a sonic dismembraner for 5 min. This insured the bacteria did not fall out of the mineral oil or saline, but remained in suspension for the time required to give all of the animals their shots.

After the adjustment period, rats were randomized into five treatment groups as follows: 1) saline/M. Butyricum suspended in slaine (non-arthritic control for drug and antigen challenge), 2) sodium narcistatin/M. Butyricum suspended in saline (non-arthritic; drug control for antigen challenge), 3) no treatment-CFA (arthritic; control for stress of injections), 4) saline-CFA (arthritic; control for drug treatment), and 5) sodium-narcistatin-CFA (arthritic) treatments.

The experiment was completed with an N=4 then repeated with an additional N=4. The data from each group in the first and repeated experiments were compared. No statistical differences between the findings from these repeat experiment were found; therefore, the data was combined to give an N=8 for each experimental group. CFA or saline/M. Butyricum injections were given on experimental day 1. Sodium narcistatin (4.5 mg/Kg/day, 250 μl) or vehicle (250 μl sterile 0.9% saline) treatments were started on day 10 and continued through day 28 post-immunization. Untreated animals were handled but received no injection. The time point to initiate drug treatments was chosen based on physical symptoms (soft tissue swelling and redness in the hind limbs) representing the time point where disease onset was confirmed.

While there is variability in the severity of disease development between the batches of adjuvant, there is very little within batch variability. All animals in each experiment were challenged with the same preparation of adjuvant. All animals in this experiment that received CFA developed arthritis from this adjuvant preparation. The arthritis disease pathology that developed in both experiments was the same in onset and severity. The arthritic animals appeared to tolerate the drug well based on no differences in body weights, grooming, feeding and drinking behavior between vehicle- and narcistatin-treated arthritic rats.

Adhesion Scoring: The adhesions were scored based on area covered and density of fibrous encasement of the spleen. Scoring was completed as follows: No gross fibrosis present on the spleen-the spleen pulls easily to the front of the abdominal cavity with a slight tug on the pancreas. Slight fibrosis visible on the surface of the spleen-spleen easily comes to front of the abdominal cavity by pulling on the pancreas-light tacking down of spleen to the loose pancreatic tissues, spleen is easily visible within the connective tissue—while one has to dissect out the spleen from these tissues—easily able to separate the connective tissue with a forceps. Slight fibrosis visible on the surface of the spleen-spleen easily comes to front of the abdominal cavity by pulling on the pancreas-light tacking down of spleen to the loose pancreatic tissues, spleen is easily visible within the connective tissue—while one has to dissect out the spleen from these tissues—easily able to separate the connective tissue with a forceps.

Moderate tacking down of the spleen within the loose pancreatic tissues—spleen does not easily come to the front of the abdominal cavity by pulling slightly on the pancreatic tissue—have to dissect out the spleen deeper in the cavity. Severe tacking down of the spleen within the loose pancreatic tissues (some connective tissue involvement of the pancreatic tissues—have to dissect out the spleen deeper in the cavity-spleen dimensions are not visible within the fibrous encasement—is tacked down to abdominal wall-spleen is removed intact from the connective tissue using a scissors and forceps.

Severe tacking down of the spleen to the pancreas, stomach, and small intestine—have to dissect out the spleen from deep in the peritoneal cavity—the connective tissue mass containing the stomach, pancreas, small intestine and spleen are tacked down to abdominal wall—spleen dimensions are not visible within the fibrous encasement—the spleen is removed in pieces from the connective tissue using a scissors and forceps.

Immune Cell Cytokine Profile Assessment: A second experiment was completed to determined whether the adhesion reducing action of sodium narcistatin could be due to altering immune cell pro-inflammatory cytokine profiles known to inhibit adhesion formation. Spleen cells were cultured from young adult Lewis rats and treated in vitro with either Con A or LPS, T or B cell mitogen, respectively. Cultures with and without Con A or LPS were then treated with sodium narcistatin (0, 1, or 10 μg/ml). Cultures were incubated for 24 h at 37° C. After 24 h, supernatants were collected and cytokines were assessed using cytokine specific ELISA assays.

Cell Culture: Spleens were aseptically removed into Hank's balanced salt solution (HBSS). The spleens were placed in a stomacher bag and homogenized for 30 seconds. Spleen cells were triturated with a 10 ml pipette then washed with an additional 10 ml of HBSS and passed through a nylon mesh (Marsh Industries) to remove the extraneous connective tissue. The collected cells were centrifuged and resuspended in 5 ml of a NH₄Cl hypotonic buffer for 3 min to lyse the red blood cells. The cells were washed 2× with 10 ml HBSS, centrifuged and resuspended into complete RPMI 1640 media supplemented with 5% fetal calf serum and 1% antibiotic/antimycotic.

Cultures for Cytokine Analysis: The prepared cells were counted using a hemocytometer, then brought to the concentration of 2×10⁶ cells/ml. Two ml of the cell preparation were plated into 24 well plates (Falcon: Oxnard, Calif.), in the absence or presence of mitogen and in placed in an incubator 7% CO2, 37° C., for 24 h. After 24 h the culture supernatants were harvested and placed in the freezer at −70° C. until ELISA assay.

Cytokine Production: Duo sets for the detection of IL-1, IL-2, IL-4, IL-10, IFN-γ and TNF-α were obtained from R & D Systems and sandwich ELISAs were used to measure the amount cytokine released into the culture media. High binding microtiter 96-well plates were pre-coated with 100 μl of capture antibody in coating buffer for IL-1, IL-2, IL-4, IL-10, IFN-γ and TNF-α (0.15 M phosphate buffer, pH 7.4). The plates were sealed with plate film (Denville Scientific: South Plainfield, N.J.) and placed at 4° C. overnight.

The plates were allowed to come to room temperature and washed with 0.1 M phosphate buffered saline-0.5% Tween 20 (PBS-Tw20). The non-specific binding was blocked using PBS-Tw20 and 1% bovine serum albumin. Standard/sample was then added to each well, the plates were sealed and incubated 2 hours at room temperature. The plates were washed three times with PBS-Tw20 and biotinylated secondary detection antibody (diluted in PBS-1% bovine serum albumin) were added to the wells and the plates incubated for 2 hours. The plates were washed 3 times with PBS-Tw20 and streptavidin/avidin enzyme conjugate (diluted in PBS-1% bovine serum albumin) was added for 1 hour. The plates were then washed 5 times with PBS-Tw20 and developed with (TMB) reagent (Pharmingen: San Diego, Calif.) for 30-45 minutes.

After color development the plates were stopped with addition of 1N sulfuric acid. Unknown sample cytokine levels were determined through comparison with a standard curve present on each plate using an ELISA reader (Ceres 900 HDI: Bio Tek Instruments Incorporated: Winooski, Vt.). The concentrations were averaged for each treatment group at each time point, expressed as mean±SEM in ng/ml, and subjected to a one-way ANOVA (p<0.05) with Bonferroni post-hoc testing.

Fibroblast Proliferation Assessment: A third experiment was completed to determined whether the adhesion reducing action of sodium narcistatin could be due to direct effects of sodium narcistatin to prevent fibroblast proliferation.

Proliferation of rat fibroblasts were assessed using standard tritiated thymidine assays as follows. Rat fibroblast cells were cultured in the presence of narcistatin in concentrations ranging from 0 to 1 mg/ml. The assay was completed in triplicate. Cells were pulsed at 24 h with 0.5 μCi 3H-Thymidine/well and incubated for 48 h at 37° C. At 48 h plates were pulled from the incubator and cells were harvested using a Brandel Cell Harvester. Filters with collected cells were washed 12× with 200 μl of ice cold distilled water). Fiberglass filers containing cells were placed into scintillation vials with 4 ml of ScintSafe Econo2 scintillation fluid. Radioactivity measured using a scintillation counter

Results

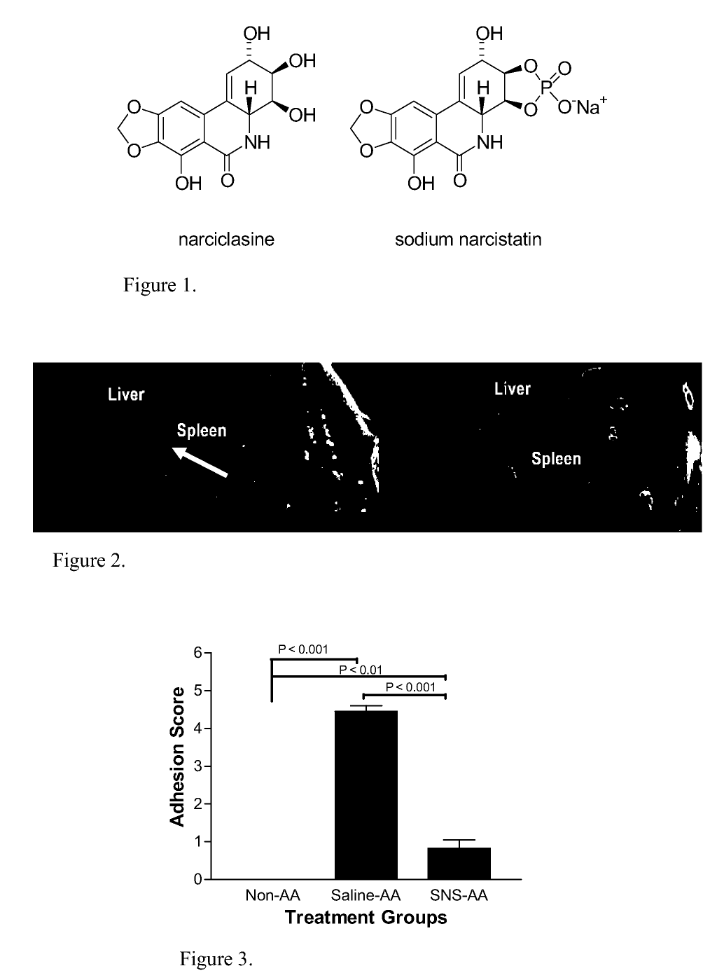

FIG. 2 is a micrograph demonstrating the extensive adhesions present surrounding the spleen in the peritoneal cavity in rats with inflammatory arthritis that were treated with the vehicle for sodium narcistatin and the lack of splenic adhesions in rats with inflammatory arthritis that were treated with sodium narcistatin. Sodium narcistatin or vehicle was administered twice daily by intraperitoneal injection. Sodium narcistatin was administered at a dose of 4.5 mg/Kg body.

Splenic adhesion development in peritoneal cavity of arthritic rats was severe in rats that were untreated or vehicle treated (FIG. 2A). In contrast, little to no adhesions were apparent in arthritic rats that were treated with sodium narcistatin (FIG. 2B).

FIG. 3 is a graph demonstrating that reduced splenic adhesion formation the peritoneal cavity in rats with inflammatory arthritis that were treated with the vehicle for sodium narcistatin and the lack of splenic adhesions in rats with inflammatory arthritis that were treated with sodium narcistatin. Sodium narcistatin or vehicle was administered twice daily by intraperitoneal injection. Sodium narcistatin was administered at a dose of 4.5 mg/Kg body. Data were obtained by a subjective scoring of adhesion formation by a single observed with the scoring completed without knowledge of the treatment administered to each animal.

Scoring assessments provide a semi-quantitative method to determine significant differences between treatment groups. For non-arthritic rats, no splenic adhesions were observed. In contrast, severe tacking down of the spleen within the loose pancreatic tissues with spleen dimensions not being visible within the fibrous encasement and the spleen being tacked down to abdominal wall-spleen was observed in vehicle treated arthritic rats. The spleen could be removed intact from the connective tissue using a scissors and forceps. Treatment with sodium narcistatin largely prevented the formation of this dense fibrous connective tissue covering over the spleen and pancreas.

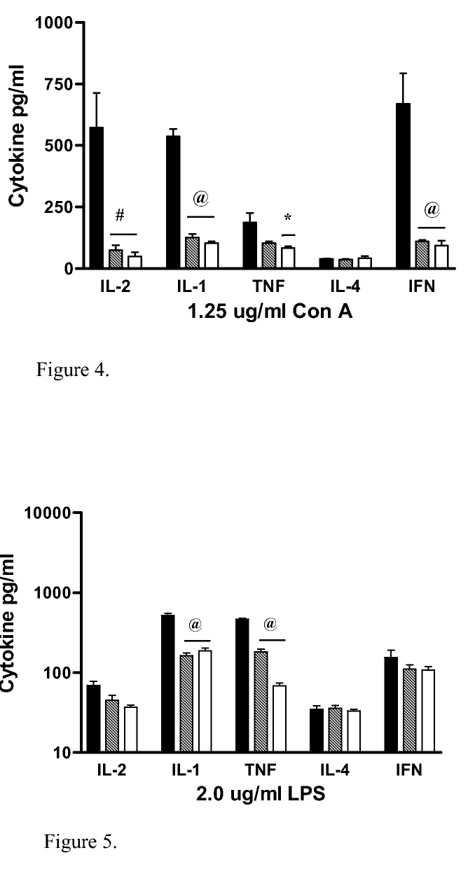

FIG. 4 is a graph demonstrating the effect of sodium narcistatin on inflammatory cytokine production by immune cells obtained from the spleen and treated with the T-cell dependent mitogen, Con A. Sodium narcistatin reduced production of IL-1 and IFN-γ, cytokines whose reduction is known to inhibit adhesion formation in the peritoneal cavity. Additionally, sodium narcistatin reduced IL-2, a cytokine that is required for clonal expansion of T helper cells involved in cell-mediated immune responses, such as injury responses that lead to the development of adhesions, fibrosis and scar tissue formation. These findings demonstrate direct anti-inflammatory effects of sodium narcistatin on immune cells.

FIG. 5 is a graph demonstrating the effect of sodium narcistatin on inflammatory cytokine production by immune cells obtained from the spleen and treated with the B-cell dependent mitogen, LPS. Sodium narcistatin reduced production of IL-1 and TNF-α, cytokines whose reduction is known to inhibit adhesion formation in the peritoneal cavity. These findings demonstrate direct anti-inflammatory effects of sodium narcistatin on immune cells.

The above data support that sodium narcistatin directly effects pro-inflammatory cytokine profiles of immune cells that have been challenged with T (FIG. 4) and B cell (FIG. 5) mitogens. Sodium narcistatin reduced production of Con-A induced IL-2, IL-1, and IFN-α and LPS-induced IL-1β and TNF-α. Reduction in TNF-α, IL-1β and INF-γ are known to reduce peritoneal adhesion formation and may be the mechanism of action for sodium narcistatin-induced inhibition of peritoneal adhesions. Alternatively, sodium narcistatin may reduce splenic adhesions in the inflammatory arthritis model by preventing fibroblast proliferation.

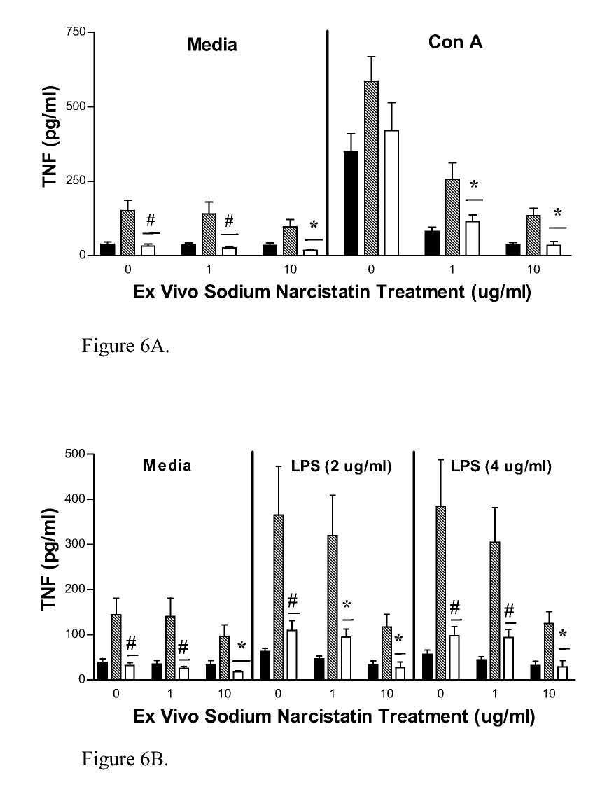

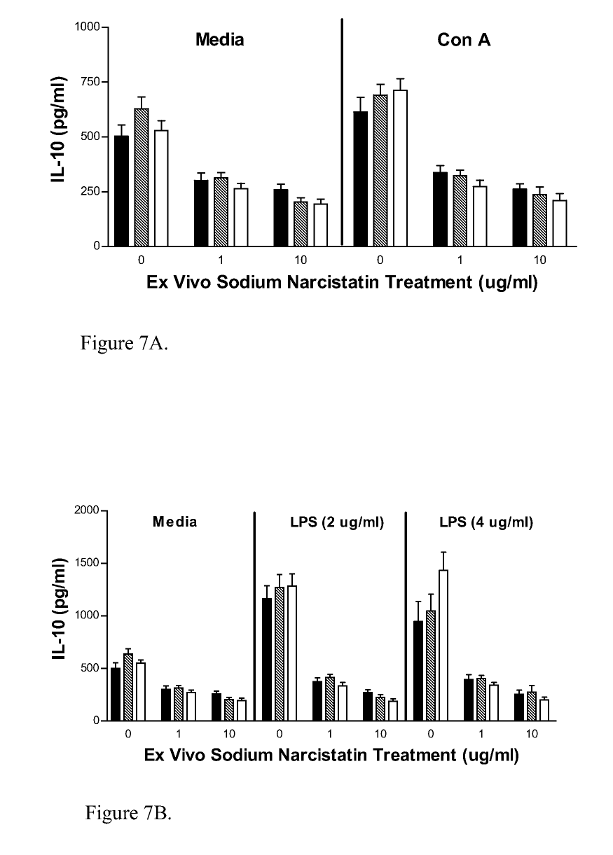

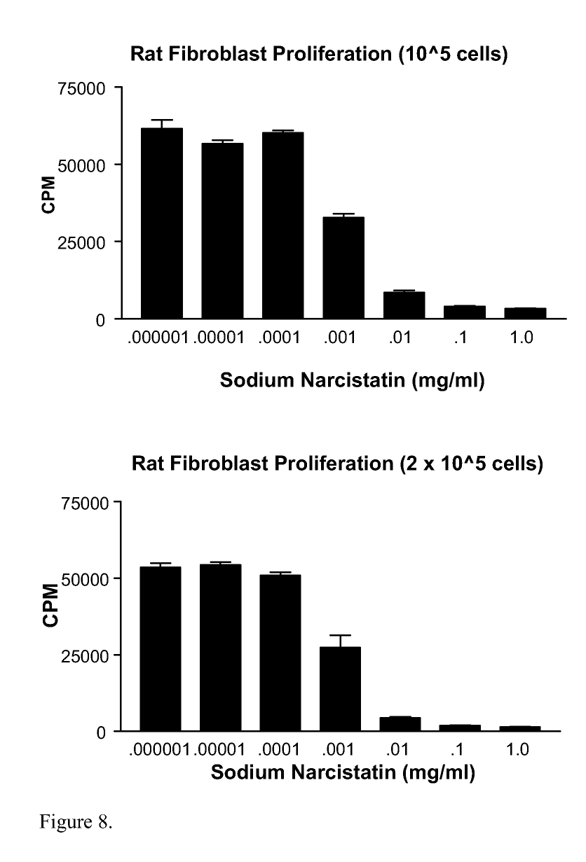

FIG. 6 is a graph demonstrating that sodium narcistatin inhibits fibroblast proliferation in vitro Sodium narcistatin was a potent inhibitor of fibroblast proliferation. Effects of sodium narcistatin on reducing fibroblast proliferation were dose dependent. Sodium narcistatin reduced fibroblast proliferation by 50% at 0.001 mg/ml and to virtually 0% at 0.01 mg/ml. These findings provide support that sodium narcistatin is an effective inhibitor of peritoneal adhesions, an effect that could be mediated by altering immune cell pro-inflammatory cytokine profiles and/or by inhibiting fibroblast proliferation.

The instant disclosure may be embodied in other forms or carried out in other ways without departing form the spirit or essential characteristics thereof. The present disclosure and enumerated examples are therefore to be considered as in all respects illustrative and not restrictive, the scope of the disclosure being indicated by the appended claims, and all equivalencies are intended to be embraced therein. One of ordinary skill in the art would be able to recognize equivalent embodiments of the instant disclosure, and be able to practice such embodiments using the teaching of the instant disclosure and only routine experimentation.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of a preferred embodiment, and best mode of the invention known to the applicant at this time of filing the application, have been presented and is intended for the purposes of illustration and description. It is not intended to be exhaustive nor limit the invention to the precise form disclosed and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated. Therefore, it is intended that the invention not be limited to the particular embodiments disclosed for carrying out the invention.

What is claimed is:

1. A method for reducing internal adhesions in an animal comprising:
administering to the animal a therapeutically effective dose of a composition comprising sodium narcistatin wherein internal adhesions are reduced.

2. The method in claim 1, wherein the therapeutically effective dose of the composition comprises 1 to 10 mg of sodium narcistatin per kilogram of body weight.

3. The method of claim 1, wherein the animal is a human.

4. The method of claim 1, wherein internal adhesions are associated with rheumatoid arthritis.

5. A method for reducing internal adhesions in an animal comprising:
administering to the animal a therapeutically effective dose of a composition comprising narciclasine wherein internal adhesions are reduced.

6. A method for reducing internal adhesions in an animal comprising:
administering to the animal a therapeutically effective dose of a composition comprising pancratastin wherein internal adhesions are reduced.

7. A composition useful for reducing internal adhesions in animals comprising sodium narcistatin.

8. The composition of claim 6, wherein the animal is a human.

9. The composition of claim 6, wherein the composition is in a form selected from the group consisting of pill, tablet, capsule, caplet, granules, powder, suppository, solution, suspension, syrup, gel and emulsion.

10. A method for preventing internal adhesions in an animal comprising administering to the animal a therapeutic dose of sodium narcistatin wherein the therapeutic dose of sodium narcistatin prevents the development of internal adhesions.
